(19) United States
(12) Patent Application Publication
(10) Pub. No.: US 2024/0076581 A1
(43) Pub. Date: Mar. 7, 2024

(54) Quat-Based Compostable and Biodegradable Pre-Moistened Cleaning and Disinfecting Wipes System with Particular Surface Frictional Characteristics

(71) Applicant: The Clorox Company, Oakland, CA (US)

(72) Inventors: Nikhil Dani, Pleasanton, CA (US); Kathryn Kicklighter, Pleasanton, CA (US); John Gelinas, Pleasanton, CA (US); Tara Cullerton, Pleasanton, CA (US)

(21) Appl. No.: 18/450,616

(22) Filed: Aug. 16, 2023

Related U.S. Application Data
(60) Provisional application No. 63/404,243, filed on Sep. 7, 2022.

(51) Int. Cl. C11D 3/48; C11D 1/83; C11D 3/20; C11D 3/30; C11D 3/43; C11D 11/00; C11D 17/04

(52) U.S. Cl. CPC C11D 3/48; C11D 1/83; C11D 3/2017; C11D 3/2068; C11D 3/2086; C11D 3/30; C11D 3/43; C11D 11/0023; C11D 17/049; C11D 1/72

(57) ABSTRACT

Pre-dosed wipes and packaged systems of such wipes including a nonwoven substrate formed from natural pulp fibers and regenerated cellulosic fibers where the natural pulp fibers are not simply present at an interior of the substrate, but are substantially homogeneously dispersed throughout the substrate, so that the natural pulp fibers are exposed on exterior faces of the wipe. As no synthetic fibers are present, the nonwoven substrate and wipe as a whole may meet any applicable biodegradability/compostability standard (e.g., ASTM D6400 or EN13432). The wipe may be substantially void of compostable synthetic polyester or other synthetic fibers, such as PHA, PLA, PVA, PVOH, PP, or PET. A cleaning composition is loaded onto the wipe. A container can be provided within which the plurality of nonwoven substrates pre-dosed with the cleaning composition are packaged.

### CROSS REFERENCE TO RELATED APPLICATIONS

This application claims the benefit of and priority to U.S. Provisional Patent Application Ser. No. 63/404,243 filed on Sep. 7, 2022, and entitled “QUAT-BASED COMPOSTABLE AND BIODEGRADABLE PRE-MOISTENED CLEANING AND DISINFECTING WIPES SYSTEM WITH PARTICULAR SURFACE FRICTIONAL CHARACTERISTICS,” which is incorporated herein by reference in its entirety.

### BACKGROUND OF THE INVENTION

### 1. The Field of the Invention

The present invention relates to cleaning wipes, e.g., pre-loaded cleaning wipes where the substrate is formed entirely from compostable (e.g., cellulosic) materials, and which are pre-loaded during manufacture with a cleaning composition. Such pre-loaded wipes may be provided within a container (e.g., packaged therein during manufacture).

### 2. Description of Related Art

Numerous cleaning wipes are available, e.g., such as CLOROX DISINFECTING WIPES. While such wipes provide good overall cleaning and disinfection characteristics, versatility, and convenience, there is a continuing need for improved cleaning wipes, as well as methods for their manufacture.

For example, a significant portion of the nonwoven substrate used in manufacturing such existing wipes is formed from materials sourced from non-renewable, petrochemical sources, where such materials (e.g., polypropylene and/or PET) are not typically biodegradable or compostable. As such wipes are typically used a single time and then disposed of, it would be advantageous for such substrates to be formed from materials that are renewable, and/or where such materials are biodegradable and/or compostable, reducing their environmental impact.

In particular, hard-surface cleaning and disinfecting wipe substrates are typically formed from synthetic polymers or blends (synthetic polymer fibers+ cellulose fibers) of different types of fibers, where the substrates are dosed with a lotion or formulation (typically referred to in the field as a lotion) that contains an antimicrobial agent that kills micro-organisms like Staph and E. Coli. The terms “lotion” and “cleaning composition” and the like are used interchangeably herein. Typically employed antimicrobial agents include quaternary ammonium compounds (also known as quaternary amines or “quats”), citric acid, lactic acid, peracetic acid, and hypochlorite. The most common types of fibers used in the substrates are polyethylene terephthalate (PET), polyethylene terephthalate/polypropylene (PET/PP), PET/rayon, and PP/wood pulp.

With the growing demand for compostable products there is a need to develop pre-moistened wipes that have similar performance characteristics to existing synthetic substrate-based materials but are also compostable. Existing compostable disinfecting and cleaning wipes are a niche market and are typically made using natural or regenerated cellulosic fiber substrates dosed with an antimicrobial formulation. There are three main classes of such compostable substrates that are commercially available:
1) 100% natural fiber (wood pulp, cotton, hemp, etc.);
2) 100% regenerated cellulose; and
3) blended substrates that combine natural fibers and regenerated cellulosic fibers at different levels.

The 100% natural fiber substrates have one major short coming—low wet strength and stiffness. The 100% regenerated substrates have shortcomings in that they are comparatively very expensive and do not have the consumer preferred “hand feel” of natural fiber substrates. All currently available blended options that combine natural and regenerated pulp fibers have one key disadvantage—they are multi-layered structures where the natural fiber is always tucked away in the middle (interior) layer(s) of the substrate, thereby preventing the substrate from having the desired “hand feel” characteristics. Such a configuration also reduces the ability of the substrate to accept and hold a texture, e.g., so as to include 3D features (texturing). Such 3D features in these types of wipe substrates could primarily be obtained by relocating the natural fibers (e.g., wood pulp) that have relatively shorter fiber lengths and are thus easier to move, to preferred regions to create peak-valley, 3D textured regions. While some flushable wipes that may include a blended substrate which is not multilayered may be available, such wipes are similar to (1) identified above, in that they exhibit very low wet strength and stiffness, making them unsuitable for use as cleaning or disinfecting wipes.

There is thus a need for a compostable hard surface cleaning and disinfecting wipe that is made using a blend of natural and regenerated cellulosic fibers that has improved characteristics.

### BRIEF SUMMARY

The present invention relates to compostable hard surface cleaning and disinfecting wipes made using a blend of natural and regenerated cellulosic fibers that exhibit improved wet strength, that exhibit an integrated, homogeneous distribution and structure of the different fiber types, where the natural and regenerated cellulosic fibers are homogeneously blended, where the natural fibers (wood pulp) “blooms” through or between the regenerated cellulose fibers so as to be exposed on the exterior surface of the wipe, so as to be available for the consumer to touch and feel (so as to provide the desired “hand feel”), and where the wipe provides particular and unusual frictional properties to facilitate improved cleaning of clingy and tenacious soils from various hard surfaces.

The present invention discloses a cleaning and disinfecting wipe that fills the need for a compostable substrate that has one or more of the below features: 1) made of compostable cellulosic fibers without any binders, synthetic resin fibers or other additives; 2) fibers are a blend of natural pulp fibers and regenerated cellulosic fibers; 3) the substrate exhibits a substantially homogeneous integrated nonwoven web structure (i.e., non-layered); 4) a significant proportion of the natural pulp fibers are accessible (i.e., exposed) on both major faces of the substrate surface; 5) relatively high wet strength; 6) formed from regenerated fibers with fiber lengths greater than 1 inch in length; and 7) the wipe as loaded with lotion exhibits a wet dynamic (sliding) coefficient of friction greater than 0.40, or greater than 0.45, and/or a wet static coefficient of friction greater than 0.5, or greater than 0.55.

Use of natural pulp fibers in combination with regenerated cellulosic fibers (e.g., lyocell, viscose, bamboo or the like) allows elimination of not only traditional polypropylene or PET synthetic, non-biodegradable fibers, but also avoids the use of so-called compostable synthetic fibers (e.g., compostable polyesters such as polyhydroxyalkanoate (PHA), or polylactic acid (PLA), as well as, polyvinyl alcohol (PVOH), or polyvinyl acetate (PVA) fibers, which are also synthetic, and typically derived from petrochemical, rather than renewable resources. No such petrochemically derived thermoplastic binder fibers (whether compostable or not) are required, or present, nor are such fibers needed to hold the substrate together. Even without such, the substrate exhibits good wet strength characteristics, similar to those of existing non-compostable wipes formed from blends of synthetic fibers and wood pulp.

Forming the entire substrate from cellulosic fibers (e.g., natural pulp fibers and regenerated cellulosic fibers) allows the nonwoven substrate to meet applicable biodegradability or compostability standards, for example, ASTM D6400, (e.g., D6400-19) or EN13432 (e.g., EN13432:2000), as substantially all components included therein may be biodegradable and/or compostable. In addition, because the pulp fiber portion of the substrate is not provided in a layered, non-homogeneous configuration, but one in which both fiber types (the pulp fibers and regenerated cellulose fibers) are exposed on the exterior faces of the wipe, the resulting wipe exhibits a combination of desired hand feel characteristics and wet strength characteristics. Such a collection of characteristics are not available within existing wipe substrates that do not provide for homogeneous distribution of pulp fibers throughout the substrate, intermingled with the regenerated cellulosic fibers in a homogeneous, or substantially homogeneous configuration.

The natural fibers (e.g., pulp fibers) and the regenerated cellulosic fibers are provided relatively homogeneously blended together, rather than present as distinct and identifiably distinct or different layers, within the substrate. In addition to the different blended fiber materials, a cleaning composition is provided, loaded onto the nonwoven substrate.

An exemplary embodiment is directed to a pre-loaded hard surface cleaning, sanitizing or disinfecting wipe comprising a nonwoven substrate as described above, e.g., comprising natural (e.g., pulp) fibers and regenerated cellulosic fibers. The pulp fibers and regenerated cellulosic fibers are substantially homogeneously distributed throughout the substrate, with natural pulp fibers exposed on exterior faces of the wipe. In an embodiment, the nonwoven substrate may be airlaid, wetlaid, carded web, through-air bonded, hydroentangled, hydroembossed, needled, or the like. A cleaning composition comprising an antimicrobial agent so as to provide sanitization or disinfection is provided pre-loaded on the nonwoven substrate, during manufacture. One particularly suitable class of antimicrobial agents include quaternary ammonium compounds, although it will be appreciated that cleaning compositions based on other antimicrobial chemistry, e.g., use of organic acids (e.g., citric, lactic, glycolic, etc.), peracetic acid or other per acids, hypochlorite, phenolic compounds (e.g., thymol, limonene, terpineol, etc.), hydrogen peroxide, or any mixture or combinations thereof may also be suitable for use. In an embodiment, the substrate as loaded has a CD/MD extensibility ratio of less than 2.

Another embodiment is directed to a pre-loaded wipe system for hard surface cleaning, sanitizing, or disinfecting wipes comprising a plurality of nonwoven substrates as described above, e.g., comprising natural (e.g., pulp, cellulose, etc.) fibers and regenerated cellulosic fibers (e.g., rayon, lyocell, viscose, modal, acetate, etc.), where the pulp fibers are homogeneously dispersed throughout the substrate, with natural pulp fibers exposed on the exterior faces of the wipe. The substrate may include 50-90% by weight natural pulp fibers and from 10-50% by weight regenerated cellulosic fibers. The cleaning composition may include 0.05%-5% by weight of one or more antimicrobial agents, 0.05%4% by weight of a surfactant, 0.1%-5% by weight of at least one glycol ether and/or alcohol solvent, and at least 90% by weight water. A container (e.g., a flex pack, cylinder, tub, or other dispensing container) may be provided within which the plurality of nonwoven substrates loaded with the cleaning composition are packaged. In an embodiment, the substrate as loaded has a CD extensibility of less than 50%.

Another embodiment is directed to a pre-loaded wipe system for hard surface cleaning, sanitizing, or disinfecting wipes comprising a plurality of nonwoven substrates as described above, e.g., consisting of natural (e.g., pulp) fibers and regenerated cellulosic fibers, where the pulp fibers are homogeneously dispersed throughout the substrate, with natural pulp fibers exposed on the exterior faces of the wipe. The cleaning composition may include one or more antimicrobial agents and at least 90% by weight water. A container (e.g., a flex pack, cylinder, tub, or other dispensing container) may be provided within which the plurality of nonwoven substrates loaded with the cleaning composition are packaged. The substrate as loaded may have a CD modulus that is greater than 3 lb_{f}/in.

In an embodiment, the antimicrobial agent comprises a quaternary ammonium compound.

In an embodiment, the as loaded wipe has a wet dynamic coefficient of friction that is greater than 0.40, or greater than 0.45 and/or a wet static coefficient of friction that is greater than 0.50, or greater than 0.55.

In an embodiment, the nonwoven substrate comprises from 50-90% by weight natural pulp fibers and from 10-50% by weight regenerated cellulosic fibers.

In an embodiment, the nonwoven substrate comprises from 60-80% by weight natural pulp fibers and from 20-40% by weight regenerated cellulosic fibers.

In an embodiment, the nonwoven substrate comprises more than 50% by weight natural pulp fibers and less than 50% by weight regenerated cellulosic fibers.

In an embodiment, the wipe has a wet (loaded) CD/MD extensibility ratio of about 1, such as less than 2, less than 1.8, less than 1.7, less than 1.6, less than 1.5, such as from 0.8 to 1.7, from 0.8 to 1.5, or from 0.8 to 1.3 The CD/MD extensibility ratio is defined as the ratio between the extensibility (or stretch) of the loaded wipe in the cross direction divided by the extensibility of the loaded wipe in the machine direction, tested according to ASTM D-5035, using the 4.2.1.4 2C-50 mm (2.0 in.) cut strip test.

In an embodiment, the natural pulp fibers comprise wood pulp fibers.

In an embodiment, the regenerated cellulose fibers comprise lyocell, viscose or the like.

In an embodiment, the substrate is substantially free or entirely free of synthetic binder fibers (e.g., PP, PE), including synthetic polyester binder fibers (e.g., PET), compostable polyester binder fibers (e.g., PHA, PLA) or other compostable binder fibers (e.g., PVA, PVOH).

In an embodiment, the substrate is substantially free of chemical adhesives, such as EVA, PVOH, or other chemical adhesives.

In an embodiment, the substrate as loaded has a CD extensibility of less than 50%, less than 40%, such as 30-35%.

In an embodiment, the substrate as loaded has a CD modulus that is greater than 3 lb_{f}/in, greater than 3.5 lb_{f}/in, or greater than 4 lb_{f}/in.

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIG. 1A shows cross-direction to machine direction (CD/MD) extensibility ratios for various tested pre-dosed wipes.

FIG. 1B shows CD extensibility for the same substrates tested in FIG. 1A.

FIGS. 2-7 are photographs of various exemplary substrate materials that were tested for various parameters.

FIG. 8 shows CD modulus values for the same pre-dosed substrates tested in FIGS. 1A-1B.

### DETAILED DESCRIPTION OF THE PREFERRED EMBODIMENTS

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more surfactants.

Unless otherwise stated, all percentages, ratios, parts, and amounts used and described herein are by weight.

Numbers, percentages, ratios, or other values stated herein may include that value, and also other values that are about or approximately the stated value, as would be appreciated by one of ordinary skill in the art. As such, all values herein are understood to be modified by the term “about”. Such values thus include an amount or state close to the stated amount or state that still performs a desired function or achieves a desired result. A stated value should therefore be interpreted broadly enough to encompass values that are at least close enough to the stated value to perform a desired function or achieve a desired result, and/or values that round to the stated value. The stated values include at least the variation to be expected in a typical manufacturing or other process, and may include values that are within 10%, within 5%, within 1%, etc. of a stated value.

Some ranges may be disclosed herein. Additional ranges may be defined between any values disclosed herein as being exemplary of a particular parameter. All such ranges are contemplated and within the scope of the present disclosure.

In the application, effective amounts are generally those amounts listed as the ranges or levels of ingredients in the descriptions, which follow hereto. Unless otherwise stated, amounts listed in percentage (“%'s”) are in weight percent (based on 100% active) of any composition.

The phrase ‘free of’ or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase ‘substantially free of’ or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions or articles described herein may be free or substantially free from any specific components not mentioned within this specification.

As used herein, “disposable” is used in its ordinary sense to mean an article that is disposed or discarded after a limited number of usage events, preferably less than 25, more preferably less than about 10, and most preferably after a single usage event. The wipes disclosed herein are typically disposable.

As used herein, the term “substrate” is intended to include any material that is used to clean an article or a surface. Examples of cleaning substrates include, but are not limited to, wipes, mitts, pads, or a single sheet of material which is used to clean a surface by hand or a sheet of material which can be attached to a cleaning implement, such as a floor mop, handle, or a handheld cleaning tool, such as a toilet cleaning device. The “substrates” as used herein may include a high coefficient of friction, (i.e., they cling to the surface, thereby providing better cleaning), and as such, may exclude personal cleansing applications, where low cling is needed. In particular, the present wipes and substrates are configured for cleaning hard surfaces. Such substrates may typically be in the form of a wipe.

Such substrates may be formed of a structure of individual fibers which are interlaid, typically in a manner that is not identifiable (e.g., a nonwoven). The nonwoven substrates, or layers used to make up such a nonwoven substrate included in the present substrates may be formed by any suitable process. As the present substrates do not employ thermoplastic fibers, some processes typically employed for nonwoven formation that rely on such thermoplastic materials may not be used (e.g., meltblown, and the like). Exemplary processes that may be used include, but are not limited to airlaid, wetlaid, carded web, through-air bonded, hydroentangled, hydroembossed, needled, or combinations thereof. Various processes for forming such nonwovens will be apparent to those of skill in the art, many of which are described in U.S. Pat. No. 7,696,109, incorporated herein by reference in its entirety. EP Applications EP992338, EP1687136, EP1861529, EP1303661, and US2004/0157524 are also herein incorporated by reference, each in its entirety. These references describe various nonwoven structures which are generally illustrative, and which may be modified by using the contemplated pulp and regenerated cellulosic fibers described herein.

The terms “wipe”, “substrate” and the like may overlap in meaning, and while “wipe” may typically be used herein for convenience, it will be appreciated that this term may often be interchangeable with “substrate”.

As used herein, “wiping” refers to any shearing action that the wipe undergoes while in contact with a target surface. This includes hand or body motion, substrate-implement motion over a surface, or any perturbation of the substrate via energy sources such as ultrasound, mechanical vibration, electromagnetism, and so forth.

The cleaning compositions dosed onto the substrate as described herein may provide sanitization, disinfection, or sterilization, other cleaning, or other treatment. As used herein, the term “sanitize” shall mean the reduction of “target” contaminants in the inanimate environment to levels considered safe according to public health ordinance, or that reduces a “target” bacterial population by significant numbers where public health requirements have not been established. By way of example, an at least 99% reduction in bacterial population within a 24-hour time period is deemed “significant.” Greater levels of reduction (e.g., 99.9%, 99.99%, etc.) are possible, as are faster treatment times (e.g., within 10 minutes, within 5 minutes, within 4 minutes, within 3 minutes, within 2 minutes, or within 1 minute), when sanitizing or disinfecting.

As used herein, the term “disinfect” shall mean the elimination of many or all “target” pathogenic microorganisms on surfaces with the exception of bacterial endospores.

As used herein, the term “sterilize” shall mean the complete elimination or destruction of all forms of “target” microbial life and which is authorized under the applicable regulatory laws to make legal claims as a “sterilant” or to have sterilizing properties or qualities.

Some embodiments may provide for at least a 2 or more log reduction (e.g., 3-log reduction, or 6-log reduction) in a bacterial population within a designated time period (e.g., 10 minutes, 5 minutes, 4 minutes, 3 minutes, 1 minute, 30 seconds, 10 seconds or the like). A 2-log reduction is equivalent to a 99% reduction, a 3-log reduction is equivalent to at least a 99.9% reduction, a 4-log reduction is equivalent to at least a 99.99% reduction, a 5-log reduction is equivalent to at least a 99.999% reduction, etc. An example of a target microbe may be Staphylococcus aureus. It will be appreciated that microefficacy can also be achieved against other target microbes, numerous examples of which will be apparent to those of skill in the art.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

Reference to ASTM or other standardized tests refers to the latest version of such standard, unless otherwise specified. Standards referenced herein are herein incorporated by reference in their entirety.

### II. Exemplary Nonwoven Substrates

In an aspect, the present invention is directed to pre-loaded nonwoven substrates, where they are specifically formed from materials that are more environmentally friendly as compared to the non-biodegradable, non-compostable materials typically employed as synthetic fibers in such wipes. The structure of the wipe may be such that the natural (e.g., pulp) fibers and the regenerated cellulosic fibers are provided with all such fibers simply blended together, e.g., in a substantially homogeneous distribution of the various fiber components, rather than present as generally separate layers (e.g., with pulp fibers sandwiched between layers of regenerated cellulosic fibers). Because the fibers are generally homogeneously distributed throughout the substrate, a significant fraction of the exterior surface (i.e., the 2 major faces of such a generally 2-dimensional wipe) defined by the wipe are formed of exposed natural pulp fibers, which “bloom” from an interior of the substrate, so as to be exposed on the exterior faces of the wipe.

By way of example, the fraction of the exterior face of either or both major faces of the wipe that are made up of exposed pulp fibers may be approximately equal to the weight fraction of pulp fibers within the substrate. For example, where pulp fibers may make up 70% by weight of the substrate, approximately 70% of the exterior surface of the wipe may exposed pulp fibers. Such assumes that the density of the pulp fibers is approximately equal to the density of the regenerated cellulose fibers.

In an embodiment, at least 30%, at least 40%, at least 50%, at least 60%, or at least 70% of the exterior surface may be made up of exposed pulp fibers. Similarly, no more than 90%, no more than 80%, or no more than 70% of the exterior surface may be made up of exposed pulp fibers.

Exposure of the natural pulp fibers on the exterior surface of the substrate serves to provide desired hand-feel characteristics, while the present wipes exhibit greater stiffness and wet strength than substrates formed from 100% natural pulp fibers (e.g., paper towel or tissue). The present substrates also provide excellent absorbency characteristics (ability to load the substrate to a desired loading ratio with a cleaning composition), even at higher loading ratios than typically employed with conventional non-compostable wipes. Because no thermoplastic fibers are present in the substrate, the melt softening and binding that occurs in other substrates that include such thermoplastic fibers to provide strong binding of the natural pulp fibers is not present in the present wipes. Rather, binding of the pulp fibers is provided through mechanical entanglement of the pulp fibers with the regenerated cellulose fibers. The strength of the substrate is a complex (and unpredictable) property, dependent on many interconnected variables and is not inherently and exclusively related to the amount of thermoplastic materials or lack thereof. For example, strength of a substrate depends on (a) the raw material (fibers), particularly the degree of crystallinity and polymer type and (b) substrate construction, including level of bonding, orientation of fibers, presence of strength additives, etc. In any case, a strong bond between the two types of fibers is provided, such that shredding, or disintegration of the substrate does not readily occur.

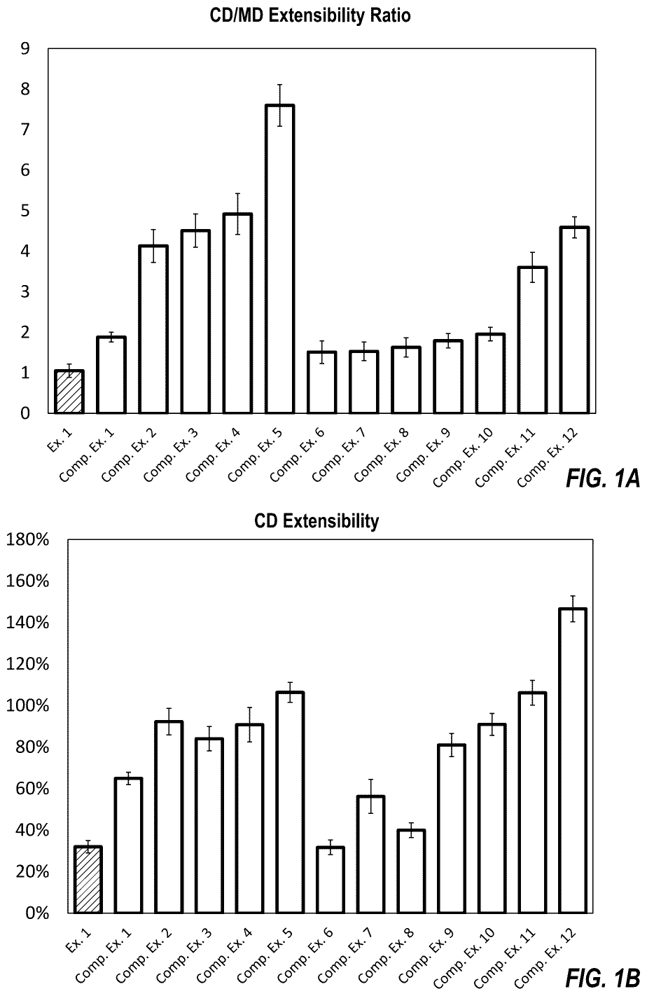

The present substrates including a homogeneous blend of natural pulp fibers with regenerated cellulose fibers have also been found to exhibit unusual extensibility characteristics, e.g., in that the extensibility in the cross-direction is similar to the extensibility in the machine-direction, such that the CD/MD extensibility ratio of the loaded wipes is close to 1. By way of example, a desirable range of extensibility for such substrates, in the cross direction (as well as the machine direction), is less than 80%, less than 70%, less than 60%, less than 50%, or less than 40%, such as between about 15% and 50%, between about 20% and 40% and between about 30% and 35%. By way of example, the extensibility for an exemplary tested substrate was about 32% in the cross direction and about 30% in the machine direction. Such relatively low extensibility values are desirable due to favorable processibility and consumer dispensing experience. FIG. 1B shows CD extensibility for the same substrates tested in FIG. 1A.

A homogeneous distribution of the two types of fibers throughout the substrate advantageously provides the same hand-feel characteristics to both faces of the wipe, without requiring the complexity associated with a 3-layer wipe. A 2-layer wipe, with different layers, would provide different hand-feel characteristics on opposite faces of the wipe, which is undesirable according to the present invention. In an embodiment, bonding of different fiber types into an integral, single substrate structure is provided without the need for any chemical adhesives, thermoplastic binder fibers, or the like.

No matter the process by which the dry nonwoven substrate is formed, once, formed, a desired cleaning composition may be loaded onto the nonwoven substrate.

Various regenerated cellulosic fibers exhibiting biodegradability and/or compostability characteristics may be used, e.g., as a minority fraction of fibers, in forming the present nonwoven substrates. While polyethylene, polypropylene, PET, PVC, polyacrylics, polyamides, polystyrenes, or the like may have been used in the past as synthetic binder fibers, many if not all such materials are not biodegradable or compostable to any significant extent, within a reasonable time frame (e.g., 5 years or less), and as such, are avoided in the present invention. Similarly, even though some compostable resin materials exist (e.g., PHA, PLA, PVA, PVOH, etc.), such materials are generally still synthetic, often derived from petrochemical or similar energy intensive sources, and typically are not as readily compostable or biodegradable as cellulosic fiber materials. Non-limiting examples of such regenerated cellulosic fibers include, but are not limited to lyocell, viscose, rayon and/or bamboo. Such regenerated cellulosic fibers may typically be of longer length than the natural pulp fibers. The present invention contemplates blends of natural pulp fibers with regenerated cellulosic fibers, so that all, or substantially all fibers present in the substrate are cellulosic.

The natural fibers may comprise any of various natural fibers. In an embodiment, such natural fibers may comprise wood pulp fibers, although other sources of pulp fibers are of course also possible (e.g., cotton, hemp, other agricultural or plant-based fibers). The natural pulp fibers may typically be of shorter length than the regenerated cellulosic fibers. By way of example, the natural pulp fibers may have fiber lengths of from 0.5 mm to 10 mm, from 1 mm to 10 mm, or from 2 mm to 10 mm, while the regenerated cellulose fibers are not continuous fibers, but are still significantly longer, such as greater than 0.5 inch (13 mm) greater than 0.75 inch (19 mm) or greater than 1 inch (25 mm), such as from 1 inch (25 mm) to 2 inches (51 mm) or more in length.

The basis weight of the nonwoven substrates may be expressed in grams per square meter (gsm), and may be, for example, no more than 200 gsm, no more than 100 gsm, such as from 5 to 80 gsm, or from 10 to 60 gsm.

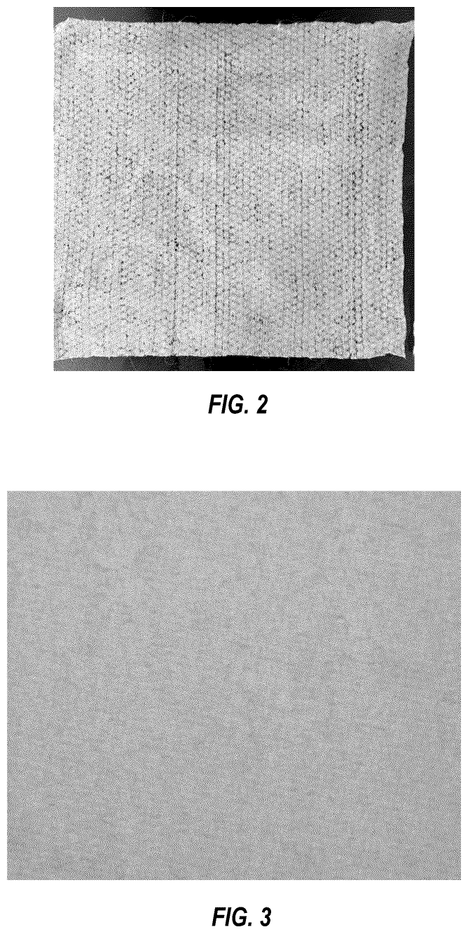

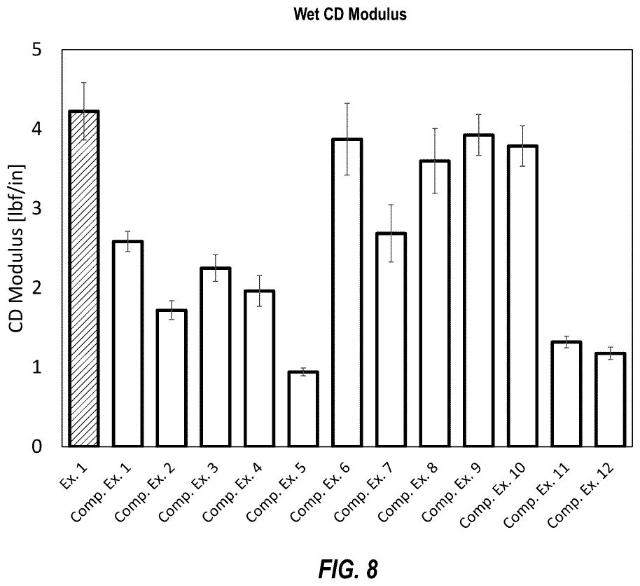

It will be appreciated that the present wipes may include any of various textures, or perhaps no texture at all. Various textures (or lack thereof) are shown in FIGS. 2-7. Additional texture examples are shown in FIGS. 1A-1D and FIGS. 7A-7F of Applicant's U.S. application Ser. No. 16/710,676 filed on Dec. 11, 2019, bearing Attorney Docket No. 510.186A (20984.12.1) herein incorporated by reference in its entirety.

Advantageously, the nonwoven substrate may consist essentially of the regenerated cellulosic fibers and the pulp fibers, so that the wipe as a whole easily meets such biodegradability/compostability standards. In an example, the wipe may be free of, or substantially free of fibers that do not meet such standards, i.e., free of polyethylene, polypropylene, PET, PVC, polyacrylics, polyamides, polystyrenes, or the like. As noted, the wipe may even be free of so-called compostable and/or biodegradable polyester resins fibers. The wipe may be free of intentionally added chemical adhesives. While no such adhesives or binders may be intentionally added as such, it will be appreciated that a so-called spin finish (e.g., a grease) may be provided on a given fiber type (e.g., regenerated cellulose) to better allow such fibers to move through an upstream process, and/or the process employed to manufacture the present substrates.

The nonwoven substrate differs from substrates used for personal care applications (e.g., for use on soft surfaces such as skin, rather than a hard surface cleaning wipe). For example, such personal care wipes are typically formed by a spunlace process, and include high polyester fiber content, so as to provide a very soft feel on skin with minimal cling. The present substrates cling significantly more to surfaces, with higher coefficient of friction and with significantly higher pulp content (and of course no polyester fibers present). Similarly, the present nonwoven substrates differ from structures used in diapers, e.g., even as layers thereof. For example, in a diaper, there is a similar interest in providing a soft smooth surface for comfortable skin contact, with very low friction. In addition, in diapers and similar products the structures are configured to absorb and retain moisture, rather than release or deliver a liquid cleaning composition, which characteristic is provided by the present wipes. In addition, typically contemplated antimicrobial agents (e.g., quats) are not typically included in a diaper or similar structure that is intended to contact skin for extended periods of time, e.g., due to skin sensitivity, etc.

The nonwoven substrates may be packaged within any desired container system. Examples of such containers include, but are not limited to flex packs, cylinders, tubs, or other containers for storage and dispensing. The wipes may typically be pulled through an orifice such as typically provided with such containers, without fear of shredding or disintegration of the wipe, due to the structural integrity provided by the substantially homogeneous blend of natural pulp fibers and regenerated cellulosic fibers.

In some embodiments, it may be desirable to provide the substrate with a lofted structure, so as to increase the bulk and thickness of the substrate, while maintaining a desired basis weight. Such lofting is aided by the fact that all fibers in the substrate are natural fibers (e.g., pulp and regenerated cellulosic fibers). Such lofting may be further enhanced, if desired, by any suitable technique that may increase the bulk and thickness of the layer, by adding lofted material thereto, which includes gaps, air pockets, and/or a fuzzy, lofted characteristic.

In an embodiment, the pulp fibers and regenerated cellulose fibers may provide for relatively low extensibility to the substrate, in both the cross-direction as well as the machine direction. As noted, extensibility in both such directions may be substantially identical, providing a wet (i.e., loaded) CD/MD extensibility ratio of approximately 1. The particular extensibility characteristics can be important in providing overall desired durability and dimensional stability to the finished wipe. A wipe that has lower extensibility characteristics, and a CD/MD extensibility ratio close to 1 is more desirable during dispensing because the stretch of the wipe is more “square”, meaning that it stretches substantially equally in both directions, and the wipe is more likely to retain its initial shape during dispensing and use.

### III. Cleaning Composition

Many cleaning composition components as known within the art may be suitable for use in the present pre-dosed wipes. In an embodiment, the cleaning composition is an aqueous, liquid composition, including at least 70%, at least 80%, at least 90% or at least 95% water by weight (e.g., 90% to 99% water). Such composition may be free, or substantially free of solid suspended materials, which may precipitate out. The composition may include 0.05% to 5% by weight of a quaternary ammonium compound or another antimicrobial agent. For example, the quaternary ammonium compound may be included from 0.05%, from 0.1%, up to 5%, up to 4%, up to 3%, up to 2%, or up to 1% by weight of the cleaning composition. In some embodiments, a glycol ether solvent and/or another solvent may be present. By way of example, such a solvent may be present at 0.05% to 5% by weight. In another embodiment, such a solvent may be included from 0.05%, from 0.1%, from 0.25%, up to 5%, up to 4%, up to 3%, up to 2%, or up to 1% by weight of the cleaning composition. Other solvents, surfactants, and various other adjuvants often included in cleaning compositions preloaded into a wipe may optionally be present. While some embodiments may include lower alcohol solvents (e.g., C₁-C₄ alcohols), the amount of such volatile solvents may be limited, e.g., to less than 10%, less than 5%, less than 3%, less than 2%, less than 1%, or less than 0.5% by weight. In some embodiments, the composition may be free of, or substantially free of, such lower alcohol or other highly volatile solvents.

Quaternary ammonium compounds have broad spectrum antimicrobial properties. A variety of different quaternary ammonium compounds can be used in the cleaning composition. Non-limiting examples of quaternary ammonium compounds typically include halides (e.g., a chloride) of alkyldimethylbenzylammonium, alkyldimethylethylbenzylammonium, alkyldimethylammonium, or the like. The alkyl groups of such quaternary ammonium compounds may typically range from C₁₂ to C₁₈. Quaternary ammonium compounds are described in more detail in U.S. Pat. No. 6,825,158, incorporated by reference herein, and will already be familiar to those of skill in the art.

Organic acids can also be used to provide antimicrobial properties. Suitable organic acids include but are not limited to, citric acid, lactic acid, glycolic acid, and any combinations thereof. By way of example, such an organic acid may be included in an amount of at least 0.1%, or at least 0.5%, up to 5%, up to 4%, up to 3%, up to 2%, up to 1.5% or up to 1% by weight of the cleaning composition.

The cleaning composition may include a solvent. In some embodiments, the solvent may include a glycol ether solvent. Exemplary glycol ether solvents include, but are not limited to, ethylene glycol monopropyl ether, ethylene glycol monobutyl ether, ethylene glycol n-hexyl ether, propylene glycol n-propyl ether, propylene glycol monobutyl ether, propylene glycol t-butyl ether, diethylene glycol monoethyl or monopropyl or monobutyl ether, di- or tri-polypropylene glycol methyl or ethyl or propyl or butyl ether, acetate and/or propionate esters of glycol ethers. Such a glycol ether solvent may be included from 0.05%, from 0.1%, from 0.25%, up to 5%, up to 4%, up to 3%, up to 2%, or up to 1% by weight of the cleaning composition.

Those of skill in the art will appreciate that any among a wide variety of surfactants (e.g., anionic, cationic, non-ionic, zwitterionic, and/or amphoteric) may be included in the cleaning composition, as desired. Where included, a surfactant may be present from 0.05%, from 0.1%, up to 10%, up to 5%, up to 4%, up to 3%, up to 2%, or up to 1% by weight of the cleaning composition. Various surfactants and other optional adjuvants are disclosed in U.S. Pat. No. 3,929,678 to Laughlin and Heuring, U.S. Pat. No. 4,259,217 to Murphy, U.S. Pat. No. 5,776,872 to Giret et al.; U.S. Pat. No. 5,883,059 to Furman et al.; U.S. Pat. No. 5,883,062 to Addison et al.; U.S. Pat. No. 5,906,973 to Ouzounis et al.; U.S. Pat. No. 4,565,647 to Llenado, and U.S. Publication No. 2013/0028990. The above patents and applications are each herein incorporated by reference in their entirety.

Examples of nonionic surfactants include, but are not limited to, alcohol ethoxylates, alcohol propoxylates, other alcohol alkoxylates including fatty (e.g., C₆, C₈, C₁₀, or C₁₂, or higher) alcohols or other constituents that have been alkoxylated to include both ethoxy and propoxy groups (EO-PO surfactants), alkyl phosphine oxides, alkyl glucosides and alkyl pentosides, alkyl glycerol esters, alkyl ethoxylates, and alkyl and alkyl phenol ethoxylates of all types, poly alkoxylated (e.g. ethoxylated or propoxylated) C₆-C₁₂ linear or branched alkyl phenols, C₆-C₂₂ linear or branched aliphatic primary or secondary alcohols, and C₂-C₈ linear or branched aliphatic glycols. Block or random copolymers of C₂-C₆ linear or branched alkylene oxides may also be suitable nonionic surfactants. Capped nonionic surfactants in which the terminal hydroxyl group is replaced by halide; C₁-C₈ linear, branched or cyclic aliphatic ether; C₁-C₈ linear, branched or cyclic aliphatic ester; phenyl, benzyl or C₁-C₄ alkyl aryl ether; or phenyl, benzyl or C₁-C₄ alkyl aryl ester may also be used. Sorbitan esters and ethoxylated sorbitan esters may also be useful nonionic surfactants. Other suitable nonionic surfactants may include mono or polyalkoxylated amides of the formula R¹CONR²R³ and amines of the formula R¹NR²R³ wherein R¹ is a C₅-C₃₁ linear or branched alkyl group and R² and R³ are C₁-C₄ alkyl, C₁-C₄ hydroxyalkyl, or alkoxylated with 1-3 moles of linear or branched alkylene oxides. Biosoft 91-6 (Stepan Co.) is an example of an alkyl ethoxylate (or alcohol ethoxylate) having a methylene chain length of C₉ to C₁₁ with an average of 6 moles of ethoxylation. An example of an alcohol ethoxylate is ECOSURF EH-9, which is more specifically an ethylene oxide-propylene oxide copolymer mono(2-ethylhexyl) ether, available from Sigma-Aldrich.

Alkylpolysaccharide nonionic surfactants are disclosed in U.S. Pat. No. 4,565,647 to Llenado, having a linear or branched alkyl, alkylphenyl, hydroxyalkyl, or hydroxyalkylphenyl group containing from 6 to 30 carbon atoms and a polysaccharide, e.g., a polyglycoside, hydrophilic group containing from 1.3 to 10 saccharide units. Suitable saccharides may include, but are not limited to, glucosides, galactosides, lactosides, and fructosides. Alkylpolyglycosides may have the formula: R²O(CnH₂ₙO)ₜ(glycosyl), wherein R² is selected from the group consisting of alkyl, alkylphenyl, hydroxyalkyl, hydroxyalkylphenyl, and mixtures thereof in which the alkyl groups contain from 10 to 18 carbon atoms; n is 2 or 3; t is from 0 to 10, and x is from 1.3 to 10.

Fatty acid saccharide esters and alkoxylated fatty acid saccharide esters may also be suitable for use in the present invention. Examples include, but are not limited to, sucrose esters, such as sucrose cocoate, and sorbitan esters, such as polyoxyethylene(20) sorbitan monooleate and polyoxyethylene(20) sorbitan monolaurate.

Phosphate ester surfactants may also be suitable. These include mono, di, and tri esters of phosphoric acid with C₄-C₁₈ alkyl, aryl, alkylaryl, alkyl ether, aryl ether and alkylaryl ether alcohols (e.g. disodium octyl phosphate).

Zwitterionic surfactants may be suitable. As zwitterionic surfactants include both a positive and negative functional group, they may also be classified as nonionic surfactants. Many such zwitterionic surfactants contain nitrogen. Examples of such include amine oxides, sarcosinates, taurates and betaines. Examples include C₈-C₁₈ alkyldimethyl amine oxides (e.g., octyldimethylamine oxide, lauryldimethylamine oxide (also known as lauramine oxide), and cetyldimethylamine oxide), C₄-C₁₆ dialkylmethylamine oxides (e.g. didecylmethylamine oxide), C₈-C₁₈ alkyl morpholine oxide (e.g. laurylmorpholine oxide), tetra-alkyl diamine dioxides (e.g. tetramethyl hexanane diamine dioxide, lauryl trimethyl propane diamine dioxide), C₈-C₁₈ alkyl betaines (e.g. decylbetaine and cetylbetaine), C₈-C₁₈ acyl sarcosinates (e.g. sodium lauroylsarcosinate), C₈-C₁₈ acyl C₁-C₆ alkyl taurates (e.g. sodium cocoylmethyltaurate), C₈-C₁₈ alkyliminodipropionates (e.g. sodium lauryliminodipropionate), and combinations thereof. Lauryl dimethyl amine oxide (Ammonyx LO) myristyl dimethyl amine oxide (Ammonyx MO), decylamine oxide (Ammonyx DO) are examples of suitable zwitterionic surfactants, available from Stepan Co.

Non-limiting examples of anionic surfactants include alkyl sulfates (e.g., C₈-C₁₈ linear or branched alkyl sulfates such as sodium lauryl sulfate (SLS), and sodium tetradecylsulfate), alkyl sulfonates (e.g., C₆-C₁₈ linear or branched alkyl sulfonates such as sodium octane sulfonate and secondary alkane sulfonates (SAS), alkyl ethoxysulfates, fatty acids and fatty acid salts (e.g., C₆-C₁₆ fatty acid soaps such as sodium laurate), and alkyl amino acid derivatives. Other examples may include sulfate derivatives of alkyl ethoxylate propoxylates, alkyl ethoxylate sulfates, alpha olefin sulfonates, C₆-C₁₆ acyl isethionates (e.g. sodium cocoyl isethionate), C₆-C₁₈ alkyl, aryl, or alkylaryl ether sulfates, C₆-C₁₈ alkyl, aryl, or alkylaryl ether methylsulfonates, C₆-C₁₈ alkyl, aryl, or alkylaryl ether carboxylates, sulfonated alkyldiphenyloxides (e.g. sodium dodecyldiphenyloxide disulfonate), and the like.

More specific examples of nonionic and/or zwitterionic surfactants include lauryl dimethyl amine oxide (Ammonyx LO), also known as lauramine oxide, myristyl dimethyl amine oxide (Ammonyx MO), decylamine oxide (Ammonyx DO), other amine oxides, any betaines, linear alcohol ethoxylates, alcohol propoxylates, alkyl polyglucosides, and combinations thereof.

The cleaning composition may optionally include and/or be used in combination with one or more additional adjuncts. The adjuncts include, but are not limited to, fragrances or perfumes, waxes, dyes and/or colorants, solubilizing materials, stabilizers, thickeners, defoamers, hydrotropes, buffers, builders, lotions and/or mineral oils, enzymes, bleaching agents, cloud point modifiers, and/or preservatives. A variety of builder detergents can be used in and/or used in combination with the cleaning composition. Such builder detergents include, but are not limited to, phosphate-silicate compounds, zeolites, alkali metal, ammonium and substituted ammonium polyacetates, mono-, di-, and tri-alkali salts of nitrilotriacetic acid, carboxylates, aluminosilicate materials, silicates, polycarboxylates, zeolites, carbonates, phosphates, bicarbonates, polyphosphates, amines, alkanolamines, aminopolycarboxylates, polyhydroxysulfonates, starch derivatives, ethylenediamine tetraacetate, and/or metal ion sequestrants (e.g., aminopolyphosphonates such as, but not limited to, ethylenediamine tetramethylene phosphonic acid and diethylene triamine pentamethylenephosphonic acid).

In one embodiment, the builder detergent includes polyacetate and/or polycarboxylate compounds. In one aspect of this embodiment, the polyacetate and/or polycarboxylate compounds include, but are not limited to, sodium, potassium, lithium, ammonium, and substituted ammonium salts of ethylenediamine tetraacetic acid, ethylenediamine triacetic acid, ethylenediamine tetrapropionic acid, diethylenetriamine pentaacetic acid, nitrilotriacetic acid, oxydisuccinic acid, iminodisuccinic acid, mellitic acid, polyacrylic acid or polymethacrylic acid and copolymers, benzene polycarboxylic acids, gluconic acid, sulfamic acid, oxalic acid, phosphoric acid, phosphonic acid, organic phosphonic acids, acetic acid, and citric acid.

In one embodiment, the buffering and pH adjusting agents, when used, include, but are not limited to, organic acids, mineral acids, alkali metal and alkaline earth salts of citrate, silicate, metasilicate, polysilicate, borate, carbonate, carbamate, phosphate, polyphosphate, pyrophosphates, triphosphates, tetraphosphates, ammonia, hydroxide, monoethanolamine, monopropanolamine, diethanolamine, dipropanolamine, triethanolamine, and/or 2-amino-2methylpropanol.

A buffering agent can be an active detergent in its own right, and/or can be a low molecular weight, organic or inorganic material used for maintaining the desired pH. The buffer can be alkaline, acidic or neutral. Non-limiting examples of buffering agents include nitrogen-containing materials (e.g., lysine; lower alcohol amines like mono-, di-, and tri-ethanolamine; tri(hydroxymethyl) amino methane; 2-amino-2-ethyl-1,3-propanediol; 2-amino-2-methyl-propanol; 2-amino-2-methyl-1,3-propanol; disodium glutamate; methyl diethanolamide; 2-dimethylamino-2-methylpropanol; 1,3-bi s (methylamine)-cyclohexane; 1,3-diamino-propanol N,N′-tetra-methyl-1,3-diamino-2-propanol; N,N-bis(2-hydroxyethyl)glycine; tris(hydroxymethyl)methyl glycine; ammonium carbamate; citric acid; acetic acid; ammonia; alkali metal carbonates; and/or alkali metal phosphates). For additional buffers that can be used, see McCutcheon's EMULSIFIERS AND DETERGENTS, North American Edition, 1997, McCutcheon Division, MC Publishing Company which is incorporated herein by reference. In yet another and/or alternative embodiment, solubilizing materials, when used, can include, but are not limited to, hydrotropes (e.g., water soluble salts of low molecular weight organic acids such as the sodium and/or potassium salts of xylene sulfonic acid). In another and/or alternative embodiment, the acids, when used, include, but are not limited to, organic hydroxy acids, citric acids, keto acid, and the like.

In still another and/or alternative embodiment, thickeners, when used, include, but are not limited to, polyacrylic acid, xanthan gum, calcium carbonate, aluminum oxide, alginates, guar gum, methyl, ethyl, clays, and/or propylhydroxycelluloses. In yet another and/or alternative embodiment, defoamers, when used, include, but are not limited to, silicones, aminosilicones, silicone blends, and/or silicone/hydrocarbon blends. In yet a further and/or alternative embodiment, bleaching agents, when used, include, but are not limited to, peracids, perborates, percarbonates, chlorine-generating substances (e.g., chloroisocyanurates hypohalite sources), hydrogen peroxide, and/or sources of hydrogen peroxide. In an embodiment, no such bleaching agents are present. In still a further and/or alternative embodiment, preservatives, when used, include, but are not limited to, mildewstats or bacteriostats, methyl, ethyl and propyl parabens, short chain organic acids (e.g., acetic, lactic and/or glycolic acids), bisguanidine compounds (e.g., Dantagard and/or Glydant) and/or short chain alcohols (e.g., ethanol and/or IPA). In one aspect of this embodiment, the mildewstats or bacteriostats include, but are not limited to, mildewstats (including non-isothiazolone compounds) include Kathon GC, a 5-chloro-2-methyl-4-isothiazolin-3-one, Kathon ICP, a 2-methyl-4-isothiazolin-3-one, and a blend thereof, and Kathon 886, a 5-chloro-2-methyl-4-isothiazolin-3-one, all available from Rohm and Haas Company; Bronopol, a 2-bromo-2-nitropropane-1,3-diol, from Boots Company Ltd.; Proxel CRL, a propyl-p-hydroxybenzoate, from ICI PLC; Nipasol M, an o-phenyl-phenol, Na+ salt, from Nipa Laboratories Ltd.; Dowicide A, a 1,2-Benzoisothiazolin-3-one, from Dow Chemical Co.; and Irgasan DP 200, a 2,4,4′-trichloro-2-hydroxydiphenylether, from Ciba-Geigy A. G.

As used herein the term “liquid” and “cleaning composition” includes, but is not limited to, solutions, emulsions, suspensions and so forth. Thus, liquids may comprise and/or contain one or more of the following: disinfectants; antiseptics; diluents; surfactants, such as nonionic, anionic, and/or cationic; waxes; antimicrobial agents; sterilants; sporicides; germicides; bactericides; fungicides; virucides; protozoacides; algicides; bacteriostats; fungistats; virustats; sanitizers; antibiotics; pesticides; and so forth. Examples of some such components are included in, but not limited to, U.S. Pat. Nos. 6,825,158; 8,648,027; 9,006,165; 9,234,165; 9,988,594; 10,219,672; 10,421,929; 10,881,262 and U.S. Publication No. 2008/003906, each of which is herein incorporated by reference in its entirety. In some embodiments, it may be possible to provide the substrates in dry form, where dosing with a selected cleaning composition may occur later (e.g., by the user). In another embodiment, the wipes may be dosed with a solid cleaning composition, where the user may add water to the wipes, at or shortly before the time of use, resulting in the dosed wipes, ready for use. Pre-dosed wipes may be preferred, e.g., as the amount and concentrations of the components in the cleaning composition can be more carefully controlled during manufacture, than may occur where the final composition may depend on the user for dilution or water addition.

With regard to pre-moistened substrates, a selected amount of liquid may be added to the container or wipes during manufacture such that the cleaning substrates contain the desired amount of liquid. The substrates are not necessarily loaded to their saturation point but are typically loaded with the cleaning composition to some ratio less than full saturation. For example, many substrates are capable of holding about 8 to 14 times their weight in liquid. For various reasons, the substrates may be loaded at a loading ratio less than saturation, e.g., less than 7:1, less than 6:1, less than 5:1, less than 4:1, such as from 1:1 to 7:1, from 2:1 to 7:1, from 3:1 to 6:1, from 4:1 to 6:1 such as about 5:1. Such a loading ratio may be somewhat higher than that typically employed when loading wipes that include synthetic fibers. For example, wipes including a significant fraction of such synthetic fibers may tend to “dump” the composition quickly. By comparison, the present substrates exhibit more of a tendency to hold the liquid composition loaded therein and provide a more uniform delivery of such composition throughout the “mileage” of the wipe. In addition, because the present substrates tend to release the loaded composition more uniformly, they also tend to provide for significantly improved “mileage” (i.e., how much surface area or length the wipe can be used on, before it becomes too dry for continued use) as compared to existing wipes that include a significant fraction of synthetic fibers.

It is important to understand and account for how the substrate materials may affect the chemistry of the cleaning composition being dosed onto the wipes. For example, it can be important to avoid or minimize unwanted chemical interactions that may inadvertently deactivate the active agents within the cleaning composition. For example, such may occur due to incompatibility between components included in the cleaning composition versus the substrate. Such interactions can be extremely complex and unpredictable. In any case, care must often be taken to minimize or at least account for incompatibilities between the included antimicrobial agent(s) (e.g., quaternary ammonium compound or otherwise) relative to substrate materials, particularly the pulp or any other non-synthetic substrate components. It is desirable that an effective amount of any given active agent not only be loaded into the wipe, but actually be released in the “squozate” from such wipe, during use. Non-limiting examples of cleaning compositions are disclosed in U.S. Pat. No. 10,843,233 to Jha et al.; U.S. Pat. No. 10,986,841 to Macnaughtan et al.; U.S. Pat. No. 5,460,833 to Andrews et al.; U.S. Pat. No. 6,221,823 to Crisanti; U.S. Pat. No. 6,346,279 to Rochon et al.; U.S. Pat. No. 6,551,980 to Wisniewski et al.; U.S. Pat. No. 6,699,825 to Rees et al.; U.S. Pat. No. 6,803,057 to Ramirez et al.; U.S. Pat. No. 6,812,196 to Rees et al.; U.S. Pat. No. 6,936,597 to Urban; U.S. Pat. No. 7,008,600 to Katsigras et al.; U.S. Pat. No. 7,070,737 to Bains et al.; U.S. Pat. No. 7,354,604 to Ramirez et al.; U.S. Pat. No. 7,598,214 to Cusack et al.; U.S. Pat. No. 7,605,096 to Tamarchio et al.; U.S. Pat. No. 7,658,953 to Bobbert; U.S. Pat. No. 7,696,143 to McCue et al.; U.S. Pat. No. 7,915,207 to Chopskie et al.; U.S. Pat. No. 8,569,220 to Gaudrealt; U.S. Pat. No. 8,575,084 to Gaudrealt; U.S. Pat. No. 10,064,409 to Hazenkamp et al.; U.S. Pat. No. 10,076,115 to Salminen et al.; U.S. Pat. No. 10,358,624 to Mitchell et al.; U.S. Publication No. 2007/0190172 to Bobbert; PCT Publication Nos. WO 99/18180 to Raso et al.; WO 99/53006 to Masotti et al.; WO 2004/067194 to Arrigoni et al.; WO 2004/104147 to Rosiello et al.; WO 2017/174959 to Convery; and EPO Publication EP 2843034 to Nedic et al., each of which is herein incorporated by reference in its entirety.

The size and shape of the wipe can vary with respect to the intended application and/or end use of the same. The cleaning substrate can have a substantially rectangular shape of a size that allows it to readily engage standard cleaning equipment or tools such as, for example, mop heads, duster heads, brush heads, mitten shaped tools for wiping or cleaning, and so forth. In another embodiment, another shape, e.g., circular, oval, or the like) may be provided.

The wipes or other cleaning substrates may be provided pre-moistened with a cleaning composition. The wet cleaning substrates can be maintained over time in a sealable container such as, for example, within a bucket or tub with an attachable lid, sealable bags, plastic pouches (e.g., “flex packs”), canisters, jars, and so forth. Desirably the wet, stacked cleaning substrates are maintained in a resealable container. The use of a resealable container is particularly desirable when using aqueous volatile liquid compositions since substantial amounts of liquid can evaporate while using the first sheets thereby leaving the remaining sheets with little or no liquid. Exemplary resealable containers and dispensers include, but are not limited to, those described in U.S. Pat. No. 10,806,309 to Azelton et al.; U.S. Pat. No. 10,278,552 to Ma et al.; U.S. Pat. No. 4,171,047 to Doyle et al., U.S. Pat. No. 4,353,480 to McFadyen, U.S. Pat. No. 4,778,048 to Kaspar et al., U.S. Pat. No. 4,741,944 to Jackson et al., U.S. Pat. No. 5,595,786 to McBride et al.; the entire contents of each of the aforesaid references are incorporated herein by reference.

Typically, the cleaning substrates are stacked and placed in the container and the liquid subsequently added thereto, all during mass manufacturing. No matter the packaging and dosing process, once manufactured and packaged, the substrate can subsequently be used to wipe a surface. The moistened cleaning substrates can be used to treat various surfaces. As used herein “treating” surfaces is used in the broad sense and includes, but is not limited to, wiping, polishing, swabbing, cleaning, washing, disinfecting, scrubbing, scouring, sanitizing, and/or applying active agents thereto.

The wipes or other cleaning substrates of the present invention can be provided in a kit form, wherein a plurality of cleaning substrates and a cleaning tool are provided in a single package.

In addition to material composition and construction, wipe or other substrate dimensions can also be used to control dosing as well as provide ergonomic appeal. In one embodiment, substrate dimensions are from about 5½ inches to about 11 inches in length, and from about 5½ inches to about 11 inches in width to comfortably fit in a hand. The substrate can have dimensions such that the length and width differ by no more than about 4 inches. Larger substrates may be provided that can be used and then folded, either once or twice, so as to contain dirt within the inside of the fold and then the wipe can be re-used. Such larger substrates may have a length from about 5½ inches to about 13 inches and a width from about 10 inches to about 13 inches. Such substrates can be folded once or twice and still fit comfortably in the hand.

Exemplary multi-layer substrates can be tested for their ability to effectively deliver an antimicrobial quaternary ammonium compound (“quat”) or other active agent to a surface during simulated cleaning. By way of example, the substrates of the present invention may be loaded with cleaning compositions including from 0.1% to 3%, such as 0.1% to 2% by weight of the quaternary ammonium compound. In an embodiment, the wipes may release at least 40%, at least 50%, at least 55%, at least 60%, or at least 65% of the quaternary ammonium compound, i.e., quaternary ammonium compound in the squozate (i.e., the cleaning composition released from the substrate) as compared to the cleaning composition before loading. The wipes may exhibit at least a 3-log reduction in a target microbe, such as Staphylococcus aureus, within a given time frame (e.g., such as 5 minutes, 4 minutes, 3 minutes, 1 minute, 30 seconds, 10 seconds, etc.).

Selection of the particular regenerated cellulosic fiber material (e.g., viscose vs. lyocell), selection of the particular pulp fibers, and the like may affect the above described release characteristics. As noted, interactions governing what fraction of the quat is released can be complex, and unpredictable. For example, in an embodiment, the selected regenerated cellulosic fiber material comprises lyocell, as lyocell may exhibit significantly improved release characteristics as compared to viscose, even though the two materials appear superficially similar to one another (i.e., both regenerated cellulosic fibers).

### IV. Examples and Data

FIG. 1A shows extensibility ratios (CD/MD) for various tested exemplary wipe materials where CD/MD extensibility ratio is defined as the ratio of the cross direction extensibility to the machine direction extensibility of 2-inch wide test strips, with a gauge length of 3 inches (e.g., ASTM D-5035 4.2.1.4 2C-50 mm (2.0 in.) cut strip test). As shown, the extensibility for comparative wipes is significantly greater than 1, often at least 2, and sometimes as high as 3-8. Table 1 below provides details relative to the various comparative wipes tested for extensibility ratio. For the CD/MD extensibility testing, the commercially available products and those products that are not commercially available were not all loaded to the same loading ratio. The compostable products that are not commercially available (e.g., Example 1, Comparative Example 2, and Comparative Example 5) were loaded to 5×. Commercially available products were tested as commercially provided. The tested examples are arranged in the Tables and Figures such that Example 1, and Comparative Examples 1-5 are most comparable, in that they include no synthetic plastic components (i.e., they are natural wipes). Comparative Examples 6-12 include synthetic fiber components. The differences between Example 1 and Comparative Examples 1-5 are readily apparent.

TABLE 1

| | | CD/MD | |
|---|---|---|---|
| | | Extensibility | St. |
| Sample | Description | Ratio | Dev. |
| Ex. 1 | Multicomponent | 1.05 | 0.16 |
| | Homogeneous Wipe | | |
| | (regenerated + pulp) 30/70 | | |
| Comp. Ex. 1 | ARM & HAMMER Wipe | 1.88 | 0.12 |
| Comp. Ex. 2 | Layered Multicomponent Wipe | 4.13 | 0.40 |
| | (regenerated + natural) 60/40 | | |
| Comp. Ex. 3 | Integrated 100% Regenerated | 4.51 | 0.41 |
| | Cellulose | | |
| Comp. Ex. 4 | METHOD Wipes | 4.92 | 0.51 |
| Comp. Ex. 5 | Layered Multicomponent Wipe | 7.6 | 0.51 |
| | (regenerated + natural) 50/50 | | |
| Comp. Ex. 6 | Layered Multicomponent Wipe | 1.51 | 0.28 |
| | (synthetic + natural fiber) | | |
| | 40/60 | | |
| Comp. Ex. 7 | TARGET UP & UP Wipe | 1.53 | 0.23 |
| Comp. Ex. 8 | LYSOL Canister Wipe | 1.63 | 0.24 |
| Comp. Ex. 9 | WALMART GREAT VALUE | 1.79 | 0.18 |
| | Wipe | | |
| Comp. Ex. 10 | Integrated Multicomponent | 1.96 | 0.17 |
| | Wipe (synthetic + regenerated) | | |
| | 70/30 | | |
| Comp. Ex. 11 | COSTCO NICE & CLEAN | 3.60 | 0.37 |
| | Wipe | | |
| Comp. Ex. 12 | LYSOL Flex Wipe | 4.59 | 0.26 |

As shown in FIG. 1A, it is unusual for a wipe to have machine direction extensibility that is approximately equal to cross-direction extensibility. The presently contemplated substrates, which include a homogeneous distribution of regenerated cellulosic fibers and natural pulp fibers, provide such characteristics. What is further interesting is that Comparative Example 2, which also includes just regenerated cellulose and natural pulp fibers, but where such are arranged in a layered configuration, with the pulp fibers interior to the regenerated cellulose fibers, does not provide even similar results. For example, the CD/MD extensibility ratio in Comparative Example 2 is about 4, far higher than about 1. Similarly, the wipe of Comparative Example 3 is formed from 100% regenerated cellulose and exhibits a CD/MD extensibility ratio of about 4 as well. By way of example, in an embodiment the CD/MD extensibility ratio may be about 1, such as less than 2, less than 1.5, less than 1.4, less than 1.3, less than 1.2, such as from 0.8 to 1.4, from 0.8 to 1.4, from 0.8 to 1.3, from 0.9 to 1.2, from 0.9 to 1.1, or from 0.95 to 1.1.

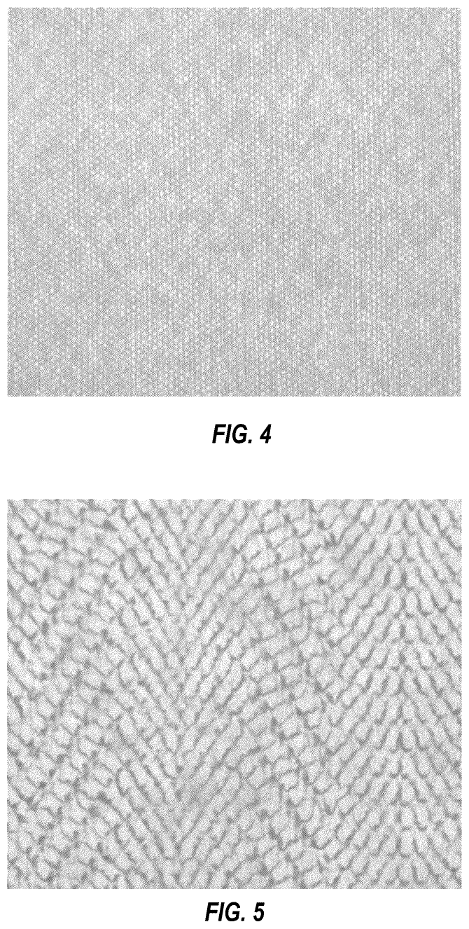

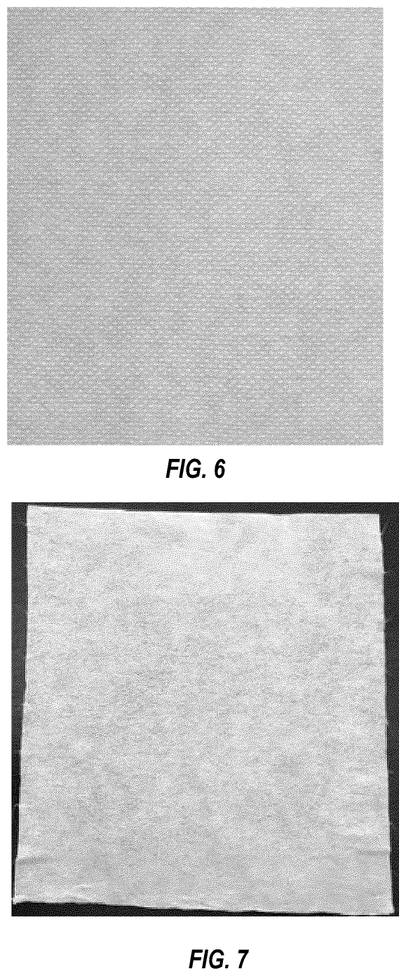

FIGS. 2-7 illustrate various exemplary wipe substrates that can either be used, or are provided by way of comparison. For example, FIG. 2 shows a wipe substrate formed from a blend of wood pulp and regenerated cellulose fibers that has been patterned to contain distinctive “dot” features. Because the pulp fibers are homogeneously distributed throughout the substrate, introduction of such texture features is possible, by moving the short pulp fibers so as to form such textured patterns. FIG. 3 shows a comparative multilayered wipe substrate made by adhering two layers of tissue. Such a wipe contains 100% wood pulp fibers, such as described in Applicant's U.S. Published Application No. 2020/0080031 to Dani et al. (425.372A/17074.141.1) which is herein incorporated by reference in its entirety. FIG. 4 shows a wetlaid, blended substrate containing wood pulp and synthetic fibers. FIG. 5 shows another blended substrate containing wood pulp and synthetic fibers, formed to have a “wavy” pattern. FIG. 6 shows a 100% PET synthetic fiber-based substrate. FIG. 7 shows a blended substrate containing different types of regenerated cellulosic fibers, such as that described in Applicant's U.S. Patent Publication No. 2023/0111060 to Frank et al. (425.388NP/17074.172.1) which is herein incorporated by reference in its entirety.

Various substrates were tested to determine their coefficient of friction characteristics. Both dynamic (sliding) and static coefficients of friction were measured. Testing included 5 replicates of each combination of the tested substrates and lotions. The results of such testing are shown in Tables 3A-3B. Such friction testing was conducted on loaded substrates at a loading ratio of 5:1, using a variety of substrates, and a variety of lotions, with the test method being based on ASTM D-1894-14 using Instron Coefficient of Friction Fixture 2810-005. The coefficient of friction was measured between the wipe and the rectangular-shaped metal table with a defined surface finish per Instron Coefficient of Friction Fixture 2810-005, where the sample wipe was wrapped taut around the sled (200 g/2.5 in square sled with foam coating).

Compositional data for the lotions is shown in Tables 2A-2C. Table 2A corresponds to lotion [A] in Tables 3A-3B. Table 2B corresponds to lotion [B] in Tables 3A-3B. Table 2C corresponds to acidic lotion [C] in Tables 3A-3B.

TABLE 2A

| Component | Function | Weight Percent |
|---|---|---|
| Water | Diluent | 90-99% |
| Glycol Ether Solvent | Solvent | 0.1-3% |
| Quaternary Ammonium Compound | Disinfectant | 0.1-2% |
| Isopropyl Alcohol | Solvent | 0.1-2% |
| Alkoxylated Alcohol | Surfactant | 0.05-1% |
| pH Adjuster | pH Adjust | 0.001-1% |
| Buffer | Buffer | 0.001-1% |
| Fragrance | Fragrance | 0.05-1% |

TABLE 2B

| Component | Function | Weight Percent |
|---|---|---|
| Water | Diluent | 90-99% |
| Glycol Ether Solvent | Solvent | 0.1-3% |
| Quaternary Ammonium Compound | Disinfectant | 0.1-2% |
| Isopropyl Alcohol | Solvent | 0.1-2% |
| Lauryl Dimethylamine Oxide | Surfactant | 0.05-1% |
| Fragrance | Fragrance | 0.05-1% |

TABLE 2C

| Component | Function | Weight Percent |
|---|---|---|
| Water | Diluent | 90-99% |
| Glycol Ether Solvent | Solvent | 0.1-3% |
| Citric Acid | Disinfectant | 0.1-2% |
| Secondary Alkane Sulfonate | Surfactant | 0.1-3% |
| Phenoxyethanol | Solvent | 0.1-2% |
| Fragrance | Fragrance | 0.05-1% |
| Defoamer | Defoam | 0.001-0.1% |

TABLE 3A

Substrate

| | | FIG. 5 | FIG. 7 | FIG. 6 | FIG. 3 |
|---|---|---|---|---|---|
| Lotion | Ex. 1 | Wipe | Wipe | Wipe | Wipe |
| Water | 0.38 | 0.26 | 0.46 | 0.25 | 0.54 |
| Lotion [A] | 0.50 | 0.30 | 0.54 | 0.30 | 0.41 |
| Lotion [B] | 0.59 | 0.36 | 0.52 | 0.32 | 0.41 |
| Lotion [C] | 0.47 | 0.30 | 0.54 | 0.23 | 0.49 |

Table 3A shows dynamic (sliding) coefficient of friction data, as average

values from the various replicates tested.

TABLE 3B

Substrate

| | | FIG. 5 | FIG. 7 | FIG. 6 | FIG. 3 |
|---|---|---|---|---|---|
| Lotion | Ex. 1 | Wipe | Wipe | Wipe | Wipe |
| Water | 0.42 | 0.27 | 0.48 | 0.30 | 0.56 |
| Lotion [A] | 0.59 | 0.37 | 0.66 | 0.44 | 0.56 |
| Lotion [B] | 0.68 | 0.43 | 0.62 | 0.45 | 0.56 |
| Lotion [C] | 0.58 | 0.36 | 0.70 | 0.37 | 0.64 |

Table 3B shows static coefficient of friction data, as average values from the various replicates tested.

The data clearly shows that wipes with substrates made with plant-based materials (e.g., Ex. 1 wipe, FIG. 7 Wipe, and FIG. 3 wipe), have higher coefficients of friction than synthetic polymer-based substrates (e.g., FIG. 5 Wipe, FIG. 6 Wipe). The wipes corresponding to Ex. 1, which include a homogeneous blend of regenerated cellulose and wood pulp fibers, arranged in a substantially homogeneous blend, with both types of fibers present at the exposed exterior face of the wipe exhibit particularly desirable hand feel and other characteristics. By way of example, the Ex. 1 wipe included 70% pulp fibers and 30% regenerated cellulose (e.g., lyocell). The way of comparison, the FIG. 7 wipe included 50% of each of two different types of regenerated cellulose fibers, and the FIG. 3 wipe included 100% pulp fibers, arranged as two separate layers adhered together.

In an embodiment, the claimed substrates may provide a wet dynamic (sliding) coefficient of friction (as pre-loaded) that is greater than 0.40, greater than 0.45, greater than 0.46, less than 0.7, less than 0.65, or less than 0.6. The claimed substrates may provide a wet static coefficient of friction (as pre-loaded) that is greater than 0.50 or greater than 0.55. Wet static coefficient of friction may be less than 0.7. Coefficient of friction values are not simply something that a designer is able to select, but such values are dictated by the numerous selections made in designing a substrate.

Testing was also performed on lotions [A]-[C] to determine their surface tension characteristics. Those of skill in the art will appreciate that the surface tension of water is about 72 dynes/cm. Employed lotions may have a surface tension of less than 50 dynes/cm, less than 40 dynes/cm, less than 35 dynes/cm, or less than 30 dynes/cm. The results for lotions [A]-[C] are shown in Table 4 below.

TABLE 4

| Lotion | Surface Tension (mN/m or dynes/cm) |
|---|---|
| Lotion [A] | 31.7 |
| Lotion [B] | 26.2 |
| Lotion [C] | 26.1 |

FIG. 8 shows wet CD modulus, for the same example materials tested in FIGS. 1A-1B. It is noted that the wet CD extensibility values shown in FIG. 1B for the present substrate are lower than all other plant-based substrates tested. The actual values are presented in Table 5 below.

TABLE 5

| | | CD | St. |
|---|---|---|---|
| | | Extensibility | Dev. |
| Sample | Description | (%) | (%) |
| Ex. 1 | Multicomponent Homogeneous | 32.0 | 3.0 |
| | Wipe (regenerated + pulp) 30/70 | | |
| Comp. Ex. 1 | ARM & HAMMER Wipe | 64.9 | 3.0 |
| Comp. Ex. 2 | Layered Multicomponent Wipe | 92.3 | 6.4 |
| | (regenerated + natural) 60/40 | | |
| Comp. Ex. 3 | Integrated 100% Regenerated | 84.0 | 5.8 |
| | Cellulose | | |
| Comp. Ex. 4 | METHOD Wipes | 90.8 | 8.3 |
| Comp. Ex. 5 | Layered Multicomponent Wipe | 106.4 | 4.8 |
| | (regenerated + natural) 50/50 | | |
| Comp. Ex. 6 | Layered Multicomponent Wipe | 31.7 | 3.6 |
| | (synthetic + natural fiber) 40/60 | | |
| Comp. Ex. 7 | TARGET UP & UP Wipe | 56.3 | 8.2 |
| Comp. Ex. 8 | LYSOL Canister Wipe | 40.0 | 3.6 |
| Comp. Ex. 9 | WALMART GREAT VALUE | 81.0 | 5.5 |
| | Wipe | | |
| Comp. Ex. 10 | Integrated Multicomponent | 90.9 | 5.3 |
| | Wipe (synthetic + regenerated) | | |
| | 70/30 | | |
| Comp. Ex. 11 | COSTCO NICE & CLEAN | 106.2 | 6.0 |
| | Wipe | | |
| Comp. Ex. 12 | LYSOL Flex Wipe | 146.6 | 6.2 |

Similarly, the wet CD modulus (FIG. 8) for the present substrates is higher than all other plant-based substrates tested. Modulus as shown in FIG. 8 is defined as the ratio of the strength of the wipe in the cross direction (expressed in pound-force) to the extensibility of the wipe in the cross direction (expressed in inches) of a 2-inch wide test strip, with a gauge length of 3 inches (e.g., ASTM D-5035 4.2.1.4 2C-50 mm (2.0 in.) cut strip test). A higher modulus equates to a higher apparent tautness of the loaded wipe. Table 6 below shows the wet CD modulus values. By way of example, in an embodiment the CD modulus may be greater than 3, greater than 3.5, greater than 4, greater than 4.05, greater than 4.1, or greater than 4.15, less than 10, less than 8, less than 6, or less than 5, such as from 4 to 6, 4 to 5, or 4 to 4.5.

TABLE 6

| | | CD | St. |
|---|---|---|---|
| | | Modulus | Dev. |
| Sample | Description | (lb_{f}/in) | (lb_{f}/in) |
| Ex. 1 | Multicomponent Homogeneous | 4.22 | 0.36 |
| | Wipe (regenerated + pulp) 30/70 | | |
| Comp. Ex. 1 | ARM & HAMMER Wipe | 2.58 | 0.13 |
| Comp. Ex. 2 | Layered Multicomponent Wipe | 1.72 | 0.12 |
| | (regenerated + natural) 60/40 | | |
| Comp. Ex. 3 | Integrated 100% Regenerated | 2.25 | 0.17 |
| | Cellulose | | |
| Comp. Ex. 4 | METHOD Wipes | 1.96 | 0.19 |
| Comp. Ex. 5 | Layered Multicomponent Wipe | 0.94 | 0.05 |
| | (regenerated + natural) 50/50 | | |
| Comp. Ex. 6 | Layered Multicomponent Wipe | 3.87 | 0.45 |
| | (synthetic + natural fiber) 40/60 | | |
| Comp. Ex. 7 | TARGET UP & UP Wipe | 2.69 | 0.36 |
| Comp. Ex. 8 | LYSOL Canister Wipe | 3.60 | 0.41 |
| Comp. Ex. 9 | WALMART GREAT VALUE | 3.92 | 0.26 |
| | Wipe | | |
| Comp. Ex. 10 | Integrated Multicomponent | 3.78 | 0.25 |
| | Wipe (synthetic + regenerated) | | |
| | 70/30 | | |
| Comp. Ex. 11 | COSTCO NICE & CLEAN | 1.32 | 0.07 |
| | Wipe | | |
| Comp. Ex. 12 | LYSOL Flex Wipe | 1.17 | 0.08 |

Without departing from the spirit and scope of this invention, one of ordinary skill can make various modifications to the invention to adapt it to various usages and conditions. As such, these changes and modifications are properly, equitably, and intended to be, within the full range of equivalence of the following claims.

1. A compostable hard surface cleaning, sanitizing or disinfecting wipe comprising:
(a) a nonwoven substrate comprising natural pulp fibers and regenerated cellulosic fibers, wherein the pulp fibers and regenerated cellulosic fibers are homogeneously distributed throughout the substrate, with natural pulp fibers exposed on exterior faces of the wipe; and
(b) a cleaning composition loaded onto said nonwoven substrate, wherein the
cleaning composition comprises an antimicrobial agent
wherein the wipe has a CD/MD extensibility ratio as loaded with the cleaning composition that is less than 2.

2. The wipe of claim 1, wherein the antimicrobial agent comprises a quaternary ammonium compound.

3. The wipe of claim 1, wherein the wipe is substantially free or entirely free of binder fibers and chemical adhesives.

4. The wipe of claim 1, wherein the wipe as loaded with the cleaning composition has:
(i) a wet dynamic coefficient of friction that is greater than 0.40; and/or
(ii) a wet static coefficient of friction that is greater than 0.5.

5. The wipe of claim 1, wherein the regenerated cellulosic fibers comprise lyocell.

6. The wipe of claim 1, wherein the nonwoven substrate comprises from 50% to 90% natural pulp fibers by weight and 10% to 50% regenerated cellulosic fibers by weight.

7. The wipe of claim 1, wherein the nonwoven substrate comprises from 60% to 80% natural pulp fibers by weight and 20% to 40% regenerated cellulosic fibers by weight.

8. The wipe of claim 1, wherein the nonwoven substrate comprises more than 50% natural pulp fibers by weight and less than 50% regenerated cellulosic fibers by weight.

9. The wipe of claim 1, wherein the wipe as loaded with the cleaning composition has a CD/MD extensibility ratio of about 1.

10. The wipe of claim 1, wherein the nonwoven substrate is substantially free or entirely free of synthetic polyester fibers or other synthetic fibers (e.g., PHA, PLA, PVA, PVOH).

11. The wipe of claim 1, wherein the natural pulp fibers comprise wood pulp fibers.

12. A compostable hard surface cleaning, sanitizing, or disinfecting wipe system packaged within a container, the system comprising:
(a) a nonwoven substrate comprising natural pulp fibers and regenerated cellulosic fibers, wherein the pulp fibers and regenerated cellulosic fibers are homogeneously distributed throughout the substrate, with natural pulp fibers exposed on exterior faces of the wipe, wherein the nonwoven substrate comprises from 50% to 90% natural pulp fibers by weight and 10% to 50% regenerated cellulosic fibers by weight; and
(b) a cleaning composition loaded onto said nonwoven substrate, wherein the cleaning composition comprises:
i. 0.05-5% by weight of one or more antimicrobial agents;
ii. 0.05-1% by weight of a surfactant;
iii. 0.1-5% by weight of at least one glycol ether or alcohol solvent; and
iv. at least 90% by weight of water; and
(c) a container within which the plurality of nonwoven substrates loaded with the cleaning composition are packaged;
wherein the CD extensibility of the substrate as loaded with the cleaning composition is less than 50%.

13. The system of claim 12, wherein the antimicrobial agent comprises a quaternary ammonium compound.

14. The system of claim 12, wherein the wipe is substantially free or entirely free of binder fibers and chemical adhesives.

15. The system of claim 12, wherein the wipe has a wet coefficient of friction that is greater than 0.45.

16. The system of claim 12, wherein the regenerated cellulosic fibers comprise lyocell.

17. The system of claim 12, wherein the wipe as loaded with the cleaning composition has a CD/MD extensibility ratio of about 1.

18. The system of claim 12, wherein the natural pulp fibers comprise wood pulp fibers.

19. A compostable hard surface cleaning or disinfecting wipe system packaged within a container, the system comprising:
(a) a nonwoven substrate consisting of natural pulp fibers and regenerated cellulosic fibers, wherein the pulp fibers and regenerated cellulosic fibers are substantially homogeneously distributed throughout the substrate, with natural pulp fibers exposed on exterior faces of the wipe; and
(b) a cleaning composition loaded onto said nonwoven substrate, wherein the cleaning composition comprises:
i. one or more antimicrobial agents;
ii. at least 90% by weight of water; and
(c) a container within which the plurality of nonwoven substrates loaded with the cleaning composition are packaged;
wherein a CD wet modulus of the nonwoven substrate is greater than 3 lb_{f}/in.

20. The system of claim 19, wherein the antimicrobial agent comprises a quaternary ammonium compound.
